(19) United States
(12) Patent Application Publication
(10) Pub. No.: US 2022/0348525 A1
(43) Pub. Date: Nov. 3, 2022

(54) METHOD FOR PREPARING NEOPENTYL GLYCOL

(71) Applicant: LG CHEM, LTD, Seoul (KR)

(72) Inventors: Sungpil YANG, Daejeon (KR); Tae Yun KIM, Daejeon (KR); Sungshik EOM, Daejeon (KR); Dong Hyun KO, Daejeon (KR); Jusang YANG, Daejeon (KR); Sangjun SHIN, Daejeon (KR)

(21) Appl. No.: 17/778,132

(22) PCT Filed: Oct. 7, 2021
(86) PCT No.: PCT/KR2021/013765

§ 371 Date: May 19, 2022

(30) Foreign Application Priority Data
Oct. 16, 2020 (KR) 10-2020-0134221

(51) Int. Cl. C07C 29/154; C07C 29/80

(52) U.S. Cl. CPC C07C 29/154; C07C 29/80

(57) ABSTRACT

A method for producing neopentyl glycol comprising a step of perforning a hydrogenation reaction by injecting a hydroxypivaldehyde (HPA) solution and hydrogen into a hydrogenation reactor, and a step of adjusting the content of H₂O contained in the hydroxypivaldehyde solutionto 6.0% by weight or less before the hydroxypivaldehyde solution being injected into the hydrogenation reactor.

### CROSS-REFERENCE TO RELATED APPLICATION(S)

The present application is a National Phase entry pursuant to 35 U.S.C. § 371 of International Application No. PCT/KR2021/013765 filed on Oct. 7, 2021, and claims priority to and the benefits of Korean Patent Application No. 10-2020-0134221 filed on Oct. 16, 2020, the disclosures of which are incorporated herein by reference in their entirety.

### FIELD OF DISCLOSURE

The present application relates to a method for producing neopentyl glycol.

### BACKGROUND

Neopentyl glycol (NPG) is a white crystalline substance with a melting point of 130° C. or higher, is used as an important intermediate for various synthetic resins, and is being widely used in industries as a raw material for various plastic powder paints, synthetic lubricants, plasticizers, surfactants, textile processing agents, etc.

Such NPG is generally produced by performing aldol condensation reaction of isobutyl aldehyde and formaldehyde to make hydroxypivaldehyde (HPA), and then reacting this HPA with hydrogen under a catalyst.

Conventionally, HPA has been hydrogenated by using a slurry-type Ni-based catalyst. In this case, crude NPG, which is a hydrogenation reaction product, contains 2,2,4-trimethyl-1,3-pentanediol (TMPD), hydroxypivalic acid NPG ester (HPNE), etc. Since TMPD and HPNE have a boiling point very similar to that of NPG, they cannot be separated from each other by simple distillation. In commercial production, HPNE is convened to NPG by saponification reaction by adding sodium hydroxide, because HPNE is unstable when distilling the reaction mixture and leads to a decrease in the yield of NPG. However, the distillation process is constrained since sodium salts of HPA or other organic acids produced by the saponification reaction promote the decomposition reaction of NPG at a high temperature of 140° C. or higher. Further, it is impossible to remove TMPD which has not been converted to a non-volatile sodium salt when performing the saponification reaction.

Therefore, efforts for producing NPG at a high yield and economically are continuously being made in the art.

### SUMMARY

The present application provides a method for producing neopentyl glycol.

An embodiment of the present application provides a method for producing neopentyl glycol, the method comprising: injecting a hydroxypivaldehyde (HPA) solution and hydrogen into a hydrogenation reactor to perform a hydrogenation reaction; and adjusting a content of H₂O contained in the hydroxypivaldehyde solution to 6.0% by weight or less before the hydroxypivaldehyde solution being injected into the hydrogenation reactor.

A method for producing neopentyl glycol according to an embodiment of the present application can slow aging of a catalyst in the hydrogenation reaction by adjusting the content of H₂O contained in the hydroxypivaldehyde solution injected into the hydrogenation reactor to 6.0% by weight or less.

Accordingly, the method for producing neopentyl glycol according to an embodiment of the present application can increase the period of use of the catalyst in the hydrogenation reaction and reduce the process cost due to catalyst purchase and catalyst replacement.

### DESCRIPTION OF DRAWINGS

FIG. 1 is a drawing schematically showing a process diagram of a method for producing neopentyl glycol according to an embodiment of the present application.

FIG. 2 is a drawing schematically showing a process diagram of a conventional method for producing neopentyl glycol.

### DETAILED DESCRIPTION

Hereinafter, the present specification will be described in more detail.

In the present specification, when a member is said to be located “on” other member, this comprises not only a case in which a member is in contact with other member but also a case in which another member exists between the two members.

In the present specification, when a part “comprises” a certain component, this means that other components may be further comprised rather than excluding other components unless there is a specific opposite description.

As described above, efforts for producing NPG at a high yield and in an economical way are continuously being made in the art.

In particular, during a process for producing neopentyl glycol, H₂O is contained at a high content of about 9 to 12% by weight in a hydroxypivaldehyde (HPA) solution, which is a raw material injected into the reactor. In the process for producing neopentyl glycol, a fixed-bed reactor (FBR) comprising a Cu/SiO₂ catalyst as a fixed bed is used. The present applicant has found out that during the process for producing neopentyl glycol, the FBR operation period can be determined by long-term stability of the Cu/SiO₂ catalyst, and H₂O contained in the hydroxypivaldehyde (HPA) solution may affect the long-term stability of the Cu/SiO₂ catalyst.

Accordingly, during the process of producing neopentyl glycol in the present application, long-term stability of a catalyst in the FBR has been intended to be secured by adjusting the content of H₂O contained in the hydroxypivaldehyde (HPA) solution injected into the reactor.

A method for producing neopentyl glycol according to an embodiment of the present application comprises a step of performing a hydrogenation reaction by injecting a hydroxypivaldehyde (HPA) solution and hydrogen into a hydrogenation reactor, and comprises a step of adjusting the content of H₂O contained in the hydroxypivaldehyde solution to 6.0% by weight or less before the hydroxypivaldehyde solution is injected into the hydrogenation reactor.

In an embodiment of the present application, based on the total weight of the hydroxypivaldehyde solution injected into the hydrogenation reactor, the content of H₂O contained in the hydroxypivaldehyde solution injected into the hydrogenation reactor may be 6.0% by weight or less, 55% by weight or less, 5.0% by weight or less, or 0. When the content range of H₂O contained in the hydroxypivaldehyde solution injected into the hydrogenation reactor is satisfied, the aging of a catalyst in the hydrogenation reaction can be slowed, and accordingly the period of use of the catalyst in the hydrogenation reaction can be increased, and the process cost due to catalyst purchase and catalyst replacement can be reduced. Further, when the content of H₂O contained in the hydroxypivaldehyde solution injected into the hydrogenation reactor exceeds 6.0% by weight, it is not preferable since long-term stability of the catalyst in a fixed-bed reactor (FBR) cannot be secured. In particular, considering the content of Si eluted, it is more preferable to adjust the content of H₂O contained in the hydroxypivaldehyde solution injected into the hydrogenation reactor to 55% by weight or less.

In an embodiment of the present application, the content of H₂O in the hydroxypivaldehyde solution may be controlled to 6.0% by weight or less using a distillation column.

The distillation column may be a single distillation column or a multi-stage distillation column. For example, the distillation column may be a tray type multi-stage distillation column, but is not limited thereto, and content known in the art may be used. Further, conditions such as temperature, pressure, etc. of the distillation column can be appropriately set by those skilled in the art so as to control the content of H₂O in the hydroxypivaldehyde solution to 6.0% by weight or less as in the embodiment of the present application.

In an embodiment of the present application, the hydroxypivaldehyde solution before the step of adjusting the content of H₂O contained in the hydroxypivaldehyde solution to 6.0% by weight or less may contain 45 to 65% by weight of hydroxypivaldehyde, 1 to 5% by weight of neopentyl glycol, 15 to 35% by weight of alcohol, 8 to 12% by weight of H₂O, and 5 to 15% by weight of a high-boiling point material. Further, the hydroxypivaldehyde solution before the step of adjusting the content of H₂O contained in the hydroxypivaldehyde solution to 6.0% by weight or less may contain 50 to 60% by weight of hydroxypivaldehyde, 1 to 3% by weight of neopentyl glycol, 20 to 30% by weight of alcohol, 8 to 11% by weight of H₂O, and 5 to 10% by weight of a high-boiling point material. The hydroxypivakdehyde solution as described above has an effect of suppressing the formation of by-products since the heat of reaction can be minimized without deteriorating the reactivity.

The alcohol may be 2-ethylhexanol(2-EH), but is not limited thereto.

The high-boiling point material may comprise one or more of hydroxypivalic hydroxypivalate (HPNE), trimethylpentanediol (IMIPD), etc., but is not limited thereto.

In an embodiment of the present application, the hydrogenation reactor may be a reactor comprising a copper-based catalyst as a fixed bed. More specifically, the copper-based catalyst may be a catalyst in which copper is supported on a silicon oxide support. Further, the hydrogenation reactor may be a fixed bed reactor (FBR) filled with the copper-based catalyst, and in this case, there is no need to separate the catalyst and a reaction product, and the reaction temperature and reaction pressure can be lowered than before so that the operation is stable and economical, and the catalyst replacement work is easy and the size of the reactor can be reduced so that there is an effect of greatly reducing the investment cost.

In an embodiment of the present application, at least a portion of a product after the hydrogenation reaction may be recirculated to the hydrogenation reactor. More specifically, a product comprising neopentyl glycol may be recirculated to the hydrogenation reactor, and heat generated in the hydrogenation reaction may be easily controlled accordingly. The hydrogenation reaction may have a recycle ratio (Feed/Recycle) of 0.1 to 8.0 kg/kg, 1 to 7 kg/kg, or 2 to 6 kg/kg, and there are effects of easily controlling the reaction heat and preventing a runaway reaction within the range. The “recycle ratio (Feed/Recycle)” refers to raw material supply flow amount to recirculation flow amount, unless otherwise specified.

In an embodiment of the present application, in the step of performing a hydrogenation reaction by injecting a hydroxypivaldehyde (HPA) solution and hydrogen into the hydrogenation reactor, the hydrogenation reaction may have a reaction temperature of 100 to 250° C., 100 to 200° C., or 100 to 180° C.

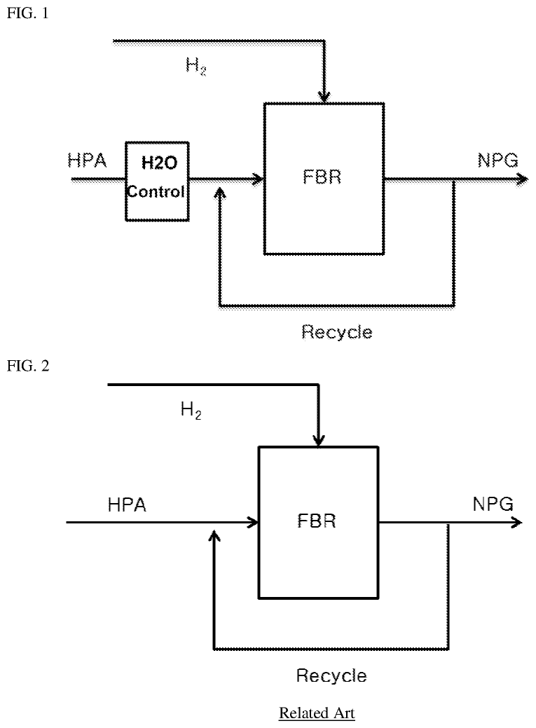

A process diagram of a method for producing neopentyl glycol according to an embodiment of the present application is schematically shown in FIG. 1 below, and a process diagram of a conventional method for producing neopentyl glycol is schematically shown in FIG. 2 below. As described in FIGS. 1 and 2 below, the method for producing neopentyl glycol according to an embodiment of the present application comprises a step of performing a hydrogenation reaction by injecting a hydroxypivakdehyde (HPA) solution and hydrogen into a hydrogenation reactor, and comprises a step of adjusting the content of H₂O contained in the hydroxypivaldehyde solution to 6.0% by weight or less before the hydroxypivaldehyde solution is injected into the hydrogenation reactor.

In the method for producing neopentyl glycol according to an embodiment of the present application, the aging of the catalyst in the hydrogenation reaction can be slowed by adjusting the content of H₂O contained in the hydroxypivaldehyde solution injected into the hydrogenation reactor to 6.0% by weight or less.

Accordingly, the method for producing neopentyl glycol according to an embodiment of the present application can increase the period of use of the catalyst in the hydrogenation reaction, and can reduce the process cost due to catalyst purchase and catalyst replacement.

Hereinafter, Examples will be given and described in detail in order to describe the present application specifically. However, Examples according to the present application may be modified in various other forms, and the scope of the present application is not to be construed as being limited to Examples described in detail below. Examples of the present application are provided to more completely explain the present application to those of ordinary skill in the art.

### EXAMPLES

### Example 1

An HPA solution (55% by weight of HPA, 2% by weight of NPG, 25% by weight of 2-ethylhexanol, 8.7% by weight of H₂O, 9.3% by weight of a high-boiling point material) was prepared.

After controlling the content of H₂O in the HPA solution to 5.49% by weight, it was injected into a fixed bed reactor (FBR) filled with a Cu/SiO₂ catalyst, and a hydrogenation reaction was carried out at 30 bar and 110° C. to produce neopentyl glycol. The content of H₂O in the HPA solution was controlled using a distillation column.

### Example 2

The production process was carried out in the same manner as in Example 1 except that the content of H₂O in the HPA solution injected into the FBR was controlled to 5.02% by weight.

### Example 3

The production process was carried out in the same manner as in Example 1 except that the content of H₂O in the HPA solution injected into the FBR was controlled to 6.0% by weight.

### Comparative Example 1

The production process was carried out in the same manner as in Example 1 except that the process of controlling the content of H₂O in the HPA solution injected into the FBR was excluded. At this time, the content of H₂O in the HPA solution injected into the FBR was 8.7% by weight.

### EXPERIMENTAL EXAMPLE

The reaction products produced in Examples and Comparative Example were analyzed by Inductively Coupled Plasma-Optical Emission Spectroscopy (ICP-OES) to measure Si concentrations, and the measurement results are shown in Table 1 below.

The ICP-OES analysis method is as follows.

1) About 10 g of samples was accurately measured in a platinum crucible.

2) The samples were heated on ahot plate to concentrate solvent components.

3) 1 mL of nitric acid was put into the samples and heated.

4) The samples were heated and dried.

5) 1 mL of concentrated nitric acid was added to the samples, and 200 μl of hydrogen peroxide was added to the samples (the process was performed 2 to 3 times).

6) When the organic materials were dissolved, 1 mL of nitric acid was put into the samples and heated.

7) When the samples were clearly dissolved, they were diluted with 10 mL of ultrapure water.

8) The samples were analyzed with ICP-OES (PERKIN-ELMER, OPTIMA 8300DV).

TABLE 1

| | Si content (ppm) |
|---|---|
| Example 1 | 2.3 |
| Example 2 | 1.3 |
| Example 3 | 3.0 |
| Comparative Example 1 | 5.4 |

As the results of Table 1 above, it can be confirmed that there is a large amount of Si elution from the Cu/SiO₂ catalyst in the process for producing neopentyl glycol as in Comparative Example 1 so that it may cause a problem in long-term stability of the catalyst.

As shown in the above results, the method for producing neopentyl glycol according to an embodiment of the present application can slow the aging of the catalyst in the hydrogenation reaction by adjusting the content of H₂O contained in the hydroxypivaldehyde solution injected into the hydrogenation reactor to 6.0% by weight or less. In particular, considering the content of Si eluted, it is more preferable to adjust the content of H₂O contained in the hydroxypivaldehyde solution injected into the hydrogenation reactor to 5.5% by weight or less.

Accordingly, the method for producing neopentyl glycol according to an embodiment of the present application can increase the period of use of the catalyst in the hydrogenation reaction, and can reduce the process cost due to catalyst purchase and catalyst replacement.

1. A method for producing neopentyl glycol, the method comprising:
performing a hydrogenation reaction by injecting a hydroxypivaldehyde (HPA) solution and hydrogen into a hydrogenation reactor; and
adjusting a content of H₂O contained in the hydroxypivaldehyde solution to 6.0% by weight or less before the hydroxypivaldehyde solution being injected into the hydrogenation reactor.

2. The method of claim 1, wherein a distillation column is used in the step of adjusting the content of H₂O contained in the hydroxypivaldehyde solution to 6.0% by weight or less.

3. The method of claim 1, wherein the hydroxypivaldehyde solution before the step of adjusting the content of H₂O contained in the hydroxypivaldehyde solution to 6.0% by weight or less contains 45 to 65% by weight of hydroxypivaldehyde, 1 to 5% by weight of neopentyl glycol, 15 to 35% by weight of alcohol, 8 to 12% by weight of H₂O, and 5 to 15% by weight of a high-boiling point material.

4. The method of claim 1, wherein the hydrogenation reactor is a reactor comprising a copper-based catalyst as a fixed bed.

5. The method of claim 4, wherein the copper-based catalyst is a catalyst in which copper is supported on a silicon oxide support.

6. The method of claim 1, wherein at least a portion of a product after the hydrogenation reaction is recirculated to the hydrogenation reactor.

7. The method of claim 1, wherein a reaction temperature of the hydrogenation reaction is in a range of 100 to 250° C.
